SEQUENCE LISTING (11 sequences)

SEQ ID NO: 1 - DNA, 662 bp, Wild barley (accession 20-05) Dhn1 coding region
gattatcgagatctgttcgtacttcgtagcaccacaatggagtaccagggtcagcacggtcacgccaccgacaaggtggaggagtacggccagcccgtggccgggcacggcggtttcaccgacgggcccacggggacgcacggagctgccggcgtcggcggcgcgcagctccaggcgacgagggatggccacaagactgacggcgtccttcgacgctccggcagccccagctccgtaagctcccgggcagatctgtcgctgcatatattgagttgtacatgtcgctccactctatcatcttaacttgctgtgcctgtgtacgtacgtatgtagtctgaggacgacggcgtgggcgggaggaggaagaaggggatgaaggagaagatcaaggagaagctccccggaggagcccacaaggacgccgcagggcagcagcagcagacggcgatggcgggcgagtacgcgggcacacacggcatggaggccaccggcgagaagaagggcgtcatggacaagatcaaggagaagcttcccggcggacagcactgagccagccatgtgtgtcttctctatgttccgacgaagctcggctagctagcatccgcagccatgcgttactacagataaataatggactctccgatatatgtcgtggttaccag

SEQ ID NO: 2 - protein, 137 aa, Wild barely
MEYQGQHGHATDKVEEYGQPVAGHGGFTDGPTGTHGAAGVGGAQLQATRDGHKTDGVLRRSGSPSSSEDDGVGGRRKKGMKEKIKEKLPGGAHKDAAGQQQQTAMAGEYAGTHGMEATGEKKGVMDKIKEKLPGGQH

SEQ ID NO: 3 - protein, 139 aa, Dicktoo (Genebank Accession No.4105103)
MEYQGQHGHATDKVEEYGQPVAGHGGFTGGPTGTHGAAGVGGAQLQATRDGHKTDGVLRRSGSSSSSSSEDDGVGGRRKKGMKEKIKEKLPGGAHKDAAGQQQQTAMAGEYAGTHGTEATGEKKGVMDKIKEKLPGGQH

SEQ ID NO: 4 - protein, 137 aa, Himalaya137 (Genebank Accession No.296198)
MEYQGQHGHATDKVEEYGQPVAGHGGFTDGPTGTHGAAGVGGAQLQATRDGHKTDGVLRRSGSSSSSEDDGVGGRRKKGMKEKIKEKLPGGAHKDAAGQQQQTAMAGEYAGTHGTEATGEKKGVMDKIKEKLPGGQH

SEQ ID NO: 5 - protein, 139 aa, Himalaya139 (Genebank Accession No.118483); identical to SEQ ID NO: 3

SEQ ID NO: 6 - protein, 139 aa, Georgie139 (Genebank Accession No.CAA66970)
MEYQGQHGHCTDKVRSTGQPVAGHGFTGGPTGTHGAAGVGGAQLQATRDGHKTDGVLRRSGSSSSSSSEDDGVGGRRKKGMKEKIKEKLPGGAHKDARAGQQQQTAMAGEIAGTHGTEATGEKKGVMDKIKEKLPGGQH

SEQ ID NO: 7 - DNA, 1089 bp, Wild barley (accession 20-05) Dhn1 promoter
gacaagcttagtgcaagcatcgtggcaacttttagcccgtaaaaaagtcgttgaaacatatcaaattatgatctagttttgaaaatcttgtcgagacggattaatgatgaaaatgtatttttaatcgaattttttatttaagagataaatatttgaagtccaaaaaaatccgttgatgccatccgtttgatgtggcaagatggatgataacgaagacgtttggaccatattgctttgtaccacatgtgcgacacttattatttgggttttgcaatgggtcggcgtttagacgaggtgttaagtgtttaatgaacaatctttctatgcattgattggtagtcattttgtacaggtccgtatatttagcatcgttttttctggatcatcataatatcctctcttctttttaattgatttgtcatatcactattttacttatgtgagacactcttagcgcatctacgcctaggtctactgggaagatgtaatcttgatcagtttggtcgttttgtgtgtttgtcagttgtgatctagatcagtcgatgattttctttttctttttttaatttcaaagatggactgacgtcttgatccttcgttttaaaaaagttagctaattcaaacatggcgttcatttatccagagaaaaaggtgaattttgcgcctacgggtcgaggagcaacgaagtttccacgtagcacgcacgctgtaccgaacatctctattcatgcaggaatgcgtggatggagtagtcatgcccttcgagacagaccgaagacgtacgtggcgccccagacgaggccgcgtggcatgcatgccgacgttgtttattggcagtagatttatttgcccacttcactccaccataatggaaccccatacatgcgcgcacacgtactccttccacctgcaaagccagcgcatcccgaactcgaacacacttacacagccagcattatataaataaccatatagctttgcacttctctccgtcgcagttcgcaacagatcagcacacttccacagccacacgtataatctgtagatcgatcactgttcctacttcgtagcaccacaatggtagatctg

SEQ ID NO: 8 - DNA, 29 bp, Artificial Sequence
  Synthetic
atcgagatctgttcgtacttcgtagcacc

SEQ ID NO: 9 - DNA, 27 bp, Artificial Sequence
  Synthetic
gctggtaaccacgacatatatcggaga

SEQ ID NO: 10 - DNA, 24 bp, Artificial Sequence
  Synthetic
gacgagggatggccacaagactga

SEQ ID NO: 11 - DNA, 24 bp, Artificial  Sequence
  Synthetic
agtaacgcatggctgcggatgcta

(19) United States
(12) Patent Application Publication
(10) Pub. No.: US 2008/0196126 A1
(43) Pub. Date: Aug. 14, 2008

(54) Transgenic Plants Containing a Dehydrin Gene

(76) Inventors: Eviatar Nevo, Haifa (IL); Weining Song, Yangling Shaanxi (CN); Xianghong Du, Yangling Shaanxi (CN)

Correspondence Address: BROWDY AND NEIMARK, P.L.L.C.;624 NINTH STREET, NW, SUITE 300, WASHINGTON, DC, 20001-5303, US

(21) Appl. No.: 11/630,168

(22) PCT Filed: Jun. 20, 2005
(86) PCT No.: PCT/IL2005/000656

§ 371 Date: Dec. 20, 2006

Related U.S. Application Data
(60) Provisional application No. 60/580,726, filed on Jun. 21, 2004.

(51) Int. Cl. C12N 15/29; C12N 15/82; A01H 5/00

(52) U.S. Cl. 800/298; 536/23.6; 435/320.1; 800/320

(57) ABSTRACT

The present invention provides a new dehydrin gene isolated from wild barley of the Negev desert capable of improving tolerance to abiotic stresses such as, drought, cold/freezing or high salinity stress in plants, and transgenic plants transformed thereby.

### FIELD OF THE INVENTION

The present invention relates to a new dehydrin gene, designated Dhn1, isolated from wild barley of the Negev desert, an extremely arid region, encoding a dehydrin 1 protein, designated DHN1, to vectors comprising it, and to transgenic plants containing said gene.

### BACKGROUND OF THE INVENTION

Worldwide, millions of people are chronically undernourished while the projected growth of human population will add almost three billions more by the year of 2050 as estimated by the United Nations. Environmental stresses have been recognized as the most important limiting factor in world crop production. A wide range of physiological and molecular responses takes place when plants are subject to abiotic stresses such as drought, salinity and low temperature.

Dehydrins (DHNs) are water-soluble lipid-associating proteins that accumulate during low-temperature or water-deficit conditions, and are thought to play a role in freezing- and drought-tolerance in plants. DHNs are among the most abundant and most frequently observed proteins induced by abiotic stresses in plants, as well as exogenous abscisic acid (Close, 1996). DHNs are also known as Group II late embryogenesis abundant (LEA) proteins which appear to be evolutionarily conserved among photosynthetic organisms in addition to yeast (Borovskii et al., 2002; Campbell and Close, 1997; Close and Lammers, 1993; Li et al., 1998; Mtwisha et al., 1998). This family of proteins shares virtually no similarity with any enzymes or proteins of known function. Structurally, they are characterized by combinations of typical domains, with the K-segment, a lysine-rich 15-amino acid consensus sequence (EKKGIMDKIKEKPLG), as the most conserved in all plants whereas the S-segment is a phosphorylatable serine-rich tract, the Y-segment an N-terminal conserved sequence and the Φ-segment repeated units rich in glycine and polar amino acids (Close, 1996, 1997). DHNs are supposed to form putative amphiphilic α-helices which are considered to bind to intracellular molecules, in particular membranes and proteins, primarily by hydrophobic interactions (Close, 1997). Detection of dehydrin can be made through antibodies prepared against the conserved sequences (Close et al., 1993). It has been shown that DHNs are associated with endomembrane systems and plasma membrane (Asghar et al., 1994; Egerton-Warburton et al., 1997; Danyluk et al. 1998) and maize DHN1 can bind to lipid vesicles that contain acidic phospholipids (Koag et al., 2003).

Although great progress has been achieved in the studies on DHNs in the last 20 years, their function has remained unclear, a situation similar to that of other LEA proteins (Wise and Tunnacliffe, 2004). Some effect of tomato le4 on improved stress tolerance was reported when introduced into yeast (Zhang et al., 2000). However, as a whole, overexpressing DHNs in plants has not improved stress tolerance significantly (Kaye et al., 1998; Welin, 1994). In contrast, engineering plant stress tolerance through regulons has fared much better compared with that through target genes like dehydrins (Jaglo-Ottosen et al., 1998; Liu et al., 1998; Kasuga et al., 1999).

Dhn genes exist as multi-gene families in plants. In cultivated barley (Hordeum vulgare), there are more than 12 Dhn genes identified and mapped in chromosomes 3H, 4H, 5H, and 6H in barley cv Dicktoo (Choi et al., 1999 and 2000). After a cluster of Dhn loci (Dhn1/2 and Dhn4a) was found to overlap the major quantitative trait locus (QTL) for winterhardiness on chromosome 7(5H) (Pan et al. 1994), several additional studies indicate that Dhn genes are associated with environmental stress tolerance (Campbell and Close, 1997). The studies of gene expression of Dhn in barley, which have been more comprehensive than other systems, reveal that the expression profiles of Dhn genes are highly variable from one Dhn gene to another, the majority are strongly drought-induced while others are cold-induced (Choi et al., 1999; Choi et al., 2000).

The loss of genetic diversity of some of the world's crops has accelerated alarmingly in recent decades with many crops becoming increasingly susceptible to diseases, pests and environmental stresses. Wild barley (H. vulgare ssp. spontaneum) is the progenitor of cultivated barley (H. vulgare) and is still widely distributed over the eastern Mediterranean rim and western Asia (Nevo, 1992). It occupies both primary and secondary man-made habitats and its distribution center lies in the Fertile Crescent (Badr et al., 2000). Its highest genetic diversity is displayed in Israel and Jordan despite the small area of these countries. In northern Israel and particularly the Eastern Galilee and Golan Heights, it comprises massive and continuously distributed central populations fading out to small, semi-isolated, and isolated populations in the xeric southern steppes and deserts (Nevo, 1992; Owuor et al., 1999; Turpeinen et al., 2001; Baek et al., 2003). Wild barley has, particularly with accessions from the desert regions, unique resistance to water stress (Nevo, 1992). As it can survive in severely drought-stressed environments, wild barley is a potential source of genetic variation to improve drought tolerance in elite barley cultivars. With the genomic sequencing advancing rapidly among crop species, gene transfer based on native or homologous genes from related lines and species are a more attractive option for crop improvement. Wild and cultivated barleys can provide a model system to investigate the molecular basis of drought tolerance as well as natural selection for adaptation to stressed environments.

Two groups pioneered manipulation of plants to increase their tolerance to environmental stresses such as cold, freezing, drought or high salinity, but both groups did it through engineering of regulatory elements, e.g. engineering of transcription factors, and not through structural genes coding for a protein.

The results of the first group (Thomashow et al., Michigan State University) are disclosed in several patents. U.S. Pat. No. 5,891,859 and U.S. Pat. No. 5,892,009 describe a gene, designated as CBF1, encoding a protein, CBF1, which binds to a region regulating expression of genes which promote cold temperature and dehydration tolerance in plants, and can be used to transform microorganisms and plants. U.S. Pat. No. 6,417,428 discloses a plant comprising a recombinant molecule comprising a polynucleotide that encodes a polypeptide that binds to a cold or dehydration transcription regulating region comprising the sequence CCG. U.S. Pat. No. 6,706,866 discloses a non-naturally occurring binding protein comprising an amino acid sequence capable of binding to a CCG regulatory sequence and an amino acid sequence which forms a transcription activation region, and plants that overexpress said proteins and exhibit increased tolerance to environmental stresses such as cold, freezing, drought or high salinity.

The results of the second group (Shinozaki et al., Japan International Research Center) are also disclosed in several patents. U.S. Pat. No. 6,495,742 and U.S. Pat. No. 6,670,528 describe a transcription factor gene encoding a protein that regulates the transcription of genes located downstream of a stress responsive element, wherein said stress is dehydration stress, low temperature stress or salt stress, and transgenic plants transformed therewith, and transgenic plants transformed with a DNA coding for the Arabidopsis Thaliana transcription factor DERB1A, that binds to a stress responsive element.

### SUMMARY OF THE INVENTION

In one aspect, the present invention relates to a dehydrin gene isolated from wild barley (accession 20-05), herein designated Dhn 1. SEQ ID NO:1 comprises the coding region of said Dhn1 gene. This gene codes for the new dehydrinl protein DHN1 of SEQ ID NO:2.

The present invention further relates to plants transformed with said Dhn1 gene and overexpressing DHN1, said plants exhibiting tolerance to abiotic stresses, such as drought, cold/freezing and high salinity stresses.

### BRIEF DESCRIPTION OF THE FIGURES

FIG. 1 depicts the construct pNEdhn of the invention. 35S, CaMV35S promoter. Dhn1, Dhn1 coding region. NOS, Nos terminator.

FIG. 2A shows a comparison of the amino acid sequences of different DHN1 proteins: the DHN1 of the invention (SEQ ID NO: 2) from wild barley (Genebank of Institute of Evolution at Haifa University, accession number 20-05) and the DNH1s from the barley cultivars Dicktoo (4105103) (SEQ ID NO: 3), Himalaya137 (296198) (SEQ ID NO: 4), Himalaya139 (118483) (SEQ ID NO: 5), and Georgie139 (CAA66970) (SEQ ID NO: 6). Dashes indicate where a sequence has been expanded to allow optimal alignment. The arrow indicates the intron position. The Y-, S-, K- and Φ segments are in boldface. FIG. 2B depicts the cladogram of DHN1 sequences derived from FIG. 2A. Alignments of the amino acid sequences were carried out with the Clustal-W and Boxshade programs.

FIGS. 3A-3C show the effects of drought stress on control and transgenic plants overexpressing DHN1. Plants were grown for 3 weeks with normal watering, withheld water for 5 weeks. Control and transgenic plants are shown under the same conditions. FIG. 3A shows wild-type plant without drought stress; FIG. 3B shows transgenic line N26 drought-stressed; FIG. 3C shows control plant drought-stressed.

FIG. 4 shows effects of severe drought stress and re-watering on transgenic plants overexpressing DHN1. Eight-week old plants with normal watering were transplanted to dry soils, re-watered 24 hours after transplanting, photograph was taken 36 hours after re-watering. Right, transgenic line N9. Left, control plant.

### DETAILED DESCRIPTION OF THE INVENTION

In one aspect, the present invention relates to an isolated DNA having a nucleotide sequence coding for the dehydrin 1 (DHN1) protein of SEQ ID NO:2.

In preferred embodiments, the isolated DNA has a nucleotide sequence selected from the group consisting of SEQ ID NO: 1, isolated DNA having a nucleotide sequence differing from SEQ. ID NO: 1 in codon sequence due to the degeneracy of the genetic code, and cDNA consisting of the nucleotide sequence coding for the dehydrin 1 (DHN1) protein of SEQ ID NO:2.

The DNA of SEQ ID NO: 1 was obtained from genomic DNA and includes the coding region of the dehydrin 1 gene, herein designated dhn1, of the wild-type barley (Genebank of Institute of Evolution at Haifa University, Israel, accession number 20-05) found in the northern Negev region of Israel. This DNA codes for the protein dehydrin 1, herein designated DHN1, of the amino acid sequence as shown in SEQ. ID NO: 2.

The present invention further relates to a chimeric DNA construct capable of expression in plant cells, comprising: (a) a DNA sequence coding for the DHN1 protein of SEQ ID NO: 2 such as cDNA coding for the DHN1 protein of SEQ ID NO: 2, and particularly SEQ ID NO: 1 and (b) DNA sequences enabling expression of the DHN1 protein in plant cells.

The invention still further relates to a recombinant vector comprising the DNA having a nucleotide sequence encoding for the dehydrin 1 (DHN1) protein of SEQ ID NO: 2 such as the DNA sequence in SEQ ID NO: 1 or cDNA coding for the DHN1 protein of SEQ ID NO: 2, in particular a recombinant expression vector comprising a chimeric gene construct in which the DNA of SEQ ID NO: 1 coding for the DHN1 protein of SEQ ID NO: 2, is operably linked to DNA sequences enabling expression of the DHN1 protein in plant cells.

The vector used to introduce the nucleic acid into the plant cell may be a plasmid, in which the DNA encoding the DHN1 protein is inserted into a unique restriction endonuclease cleavage site. The DNA is inserted into the vector using standard cloning procedures readily known in the art. This generally involves the use of restriction enzymes and DNA ligases, as described, for example, by Sambrook et al. (1989). The resulting plasmid which includes nucleic acid encoding the DHN1 protein can then be used to transform a plant cell (See generally, Gelvin and Schilperoort, 1994).

For plant transformation, the plasmid preferably also includes a selectable marker for plant transformation. Commonly used plant selectable markers include the kanamycin-resistance gene, kanamycin phosphotransferase II (nptII) gene, the hygromycin-resistance gene, hygromycin phosphotransferase (hpt) gene, the phosphinothricin acetyl transferase gene (bar), the 5-enolpyruvylshikimate-3-phosphate synthase (EPSPS) gene, neomycin 3′-O-phosphotransferase (npt II) gene, or acetolactate synthase (ALS) gene. In preferred embodiments of the invention, the selectable marker is the hpt or nptII gene, that allow selection of the transformants with hygromycin or kanamycin, respectively.

The plasmid may also include a reporter gene that, upon expression, provides a clear indication that genetic transformation did take place. Commonly used reporter genes are beta-glucuronidase (GUS), luciferase and green fluorescent protein (GFP). Reporter genes are often placed downstream of the promoter region and in the proximity of the gene of interest to ensure that they are expressed together and not separated by crossover events.

The plasmid preferably also includes suitable promoters for expression of the nucleic acid encoding the DHN1 protein and for expression of the selectable marker gene. The cauliflower mosaic virus 35S (35S CaMV) promoter is commonly used for plant transformation, as well as the rice actin 1 (Act1), the Ubiquitin 1 (Ubi1), the alpha-amylase gene promoter, and promoters of genes induced by stress. In one preferred embodiment, the promoter used in the present invention is the 35S CaMV promoter. Also the dhn1 promoter of the whole dhn1 gene, of the sequence of SEQ ID NO:7, can be used in the present invention. In the plasmid, the nucleic acid encoding the DHN1 protein may be under the control of one promoter and the marker gene may be under control of the same or of a different promoter.

For plant transformation, the plasmid also preferably includes a nucleic acid molecule encoding a 3′ terminator such as that from the 3′ non-coding region of genes encoding a proteinase inhibitor, actin, or nopaline synthase (nos). In a preferred embodiment, the plasmid of the present invention includes the nopaline synthase (NOS) terminator.

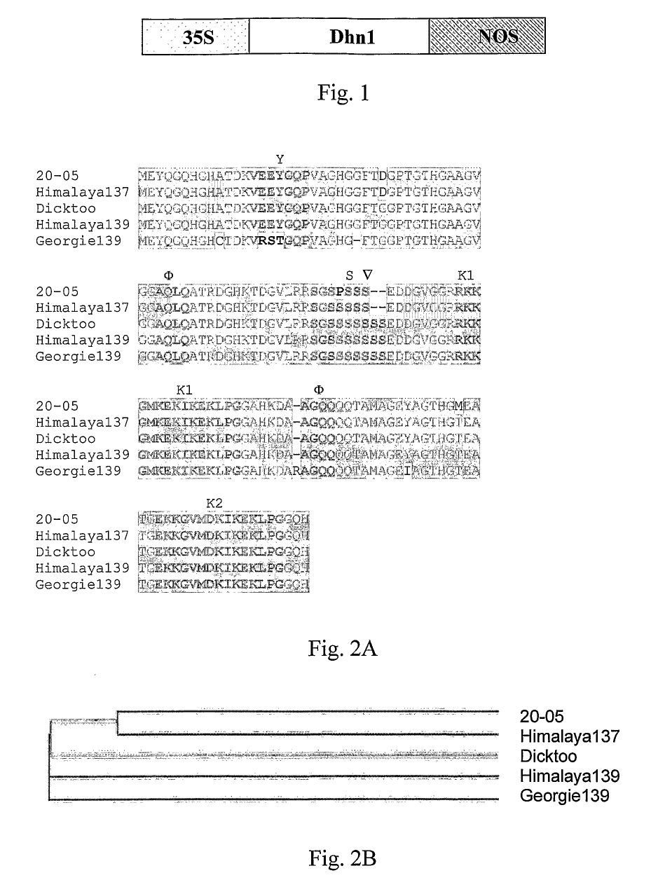

In one preferred embodiment of the invention, the recombinant vector is represented by the construct of FIG. 1.

The plasmid is preferably a binary vector in which the genes of interest are inserted within the T-DNA borders. Examples of such vectors that can be used in the present invention are vectors obtainable from commercial sources such as the pCambia 1301, the pBI121, which contains a low-copy RK2 origin of replication, the neomycin phosphotransferase (nptII) marker gene with a nopaline synthase (NOS) promoter and a NOS 3′ polyadenylation signal, the pBI101 and functionally similar vectors described by Becker et al. (1992), and the pPZPY112 vector.

For the transformation of the plants, any method suitable for transformation of cereals can be used such as Agrobacterium-mediated transformation or particle bombardment (also known as biolistic transformation).

In the Agrobacterium-mediated transformation, plant cells are contacted with an inoculum of the bacteria transformed with the plasmid comprising the gene that encodes for the DHN1 protein of the invention, for example by inoculating the plant cells with a suspension of the transformed bacteria. Suitable bacteria from the genus Agrobacterium that can be utilized to transform plant cells include the species Agrobacterium rhizogenes and Agrobacterium tumefaciens, preferably A. tumefaciens strains LBA4404 or EHA105. Agrobacterium spp. are transformed with the plasmid by conventional methods well-known in the art.

Using the floral dip transformation protocol, the A. tumefaciens strain carrying the Dhn1 gene in the binary vector is grown in a growth medium in the presence of antibiotic, e.g. kanamycin, to select for bacterial cells harboring the binary plasmid. The bacteria are spun down and resuspended in a 5% sucrose solution, followed by the addition of a surfactant, e.g. Silwet L-77. After mixing, the plants (after flowering) are dipped into the bacteria solution and kept under a dome or cover for 16-24 hours. The seeds are recovered and putative transformants are selected by plating the sterilized seeds on an antibiotic, e.g. hygromycin, and transplanting the putative transformants to soil. For higher rates of transformation, plants may be dipped two or three times at seven day intervals.

In cereals, the most commonly used tissue for transformation of wheat and barley has been the immature embryo from the developing grain (Weeks et al., 1993; Nehra et al., 1994; Becker et al., 1994; Wan and Lemaux, 1994; Barcelo and Lazzeri, 1995).

For the transformation of cereal plants, direct insertion of the DNA of interest may be carried out using the particle bombardment (biolistics) technique. According to this approach, rapidly propelled tungsten or gold microprojectiles (which are smaller than the plant cells) coated with the DNA of interest are blasted into cells. After the target tissue is bombarded with the DNA-coated particles under vacuum, the DNA of interest disperses from the particles within the cells and then integrates into their genome (Harwood et al., 2000). In an alternative, the particle bombardment technique can be combined with the Agrobacterium technique to facilitate transformation (Tingay et al., 1997; Cheng et al., 1997)

The invention further relates to a transgenic plant transformed with a DNA coding for the DHN1 protein of SEQ ID NO: 2 such as SEQ ID NO: 1 or cDNA coding for the DHN1 protein of SEQ ID NO: 2, said DNA being operably linked to DNA sequences enabling expression of the DHN1 protein in plant cells and subsequent improvement of tolerance of the plant to abiotic stresses, such as drought, high salinity and cold/freezing stresses.

Thus, the invention includes a transgenic plant which contains in its cells a chimeric gene construct capable of expression in plant cells, comprising: (a) a DNA sequence of SEQ ID NO: 1 coding for the DHN1 protein of SEQ ID NO: 2, and (b) DNA sequences enabling expression of the DHN1 protein in plant cells.

The transgenic plant according to the invention include, without being limited to, cereals such as wheat, barley, corn, rice, oat, and forage and turf grasses, or any other food crop species such as beans, including soybeans, peas, tomatoes, and oilseed rape.

The invention will now be illustrated by the following non-limiting examples.

### EXAMPLES

### Materials and Methods

(i) Plant material The wild barley accession 20-05 from Sde Boqer (Negev Highlands, Israel) was originally collected by Prof. Eviatar Nevo and maintained at the Institute of Evolution, University of Haifa (Israel). Arabidopsis thaliana ecotype Columbia was used for genetic transformation.

(ii) Isolation of genomic DNA. The Genomic DNA of wild barley was isolated using a method based on Weining and Langridge (1991) with modifications (Song and Henry, 1995). DNA was isolated on a small scale from ˜200 mg of young leaves. The leaves were grounded to a powder in microflge tubes under liquid nitrogen. The powder was then mixed with 0.6 ml of extraction buffer (2% sarkosyl (sodium lauryl sarcosinate), 0.1 M Tris-HCl, 10 mM EDTA, pH8.0) and subsequently, with 0.6 ml of phenol/chloroform (1:1). The whole mixture was shaken for 20-30 s and the aqueous phase recovered after centrifugation. The phenol/chloroform extraction was repeated and the DNA precipitated with ethanol.

(iii) Plant Transformation. The putative open reading frame of the Dhn1 gene (SEQ ID NO:1) in wild barley accession 20-05 was amplified by PCR using primers 5′-atcgagatctgttcgtacttcgtagcacc-3′ (SEQ ID NO:8) and 5′-gctggtaaccacgacatatatcggaga-3′ (SEQ ID NO:9). PCR conditions were as follows: 94° C. for 3 min, followed by 37 cycles of 94° C. for 30 s, 64° C. for 30 s, 72° C. for 1 min, and 72° C. for 5 min. PCR products were cloned into an pGEM®-T Easy Vectors (Promega) and confirmed by sequence analysis. The Dhn1 gene was excised using NcoI and Bst EII and the insert was ligated into the BamHI-SacI restricted pCambia 1301 vector between the CaMV 35S promoter and the nopaline synthase (NOS) terminator sequence of A. tumefaciens. The chimeric construct 35SCaMV-Dhn1-NOS (FIG. 1) was introduced into A. tumefaciens strain EHA105.

Arabidopsis thaliana plants were transformed according to the floral dip transformation protocol of Clough and Bent (1998) with A. tumefaciens carrying the construct. A. tumefaciens carrying construct pNEdhn (FIG. 1, 35SCaMV-Dhn1-NOS) was grown at 28° C. in LB with kanamycin to OD₆₀₀=0.8 and harvested in 5% sucrose solution with 0.05% (500 μl/L) Silwet L-77 before Arabidopsis plants were dipped. Selection of putative transformants was performed as described by Clough and Bent (1998). To select for transformants, sterilized seeds were suspended in 0.1% (w/v) sterile agarose and plated on 20 μg mL⁻¹ hygromycin. Transgenic lines resistant to hygromycin were transferred to pots and moved into a greenhouse. A small leaf sample from these transgenic plants was used to isolate genomic DNA for monitoring the presence of wild barley Dhn1 sequence by PCR (primer sequences: 5′-gacgagggatggccacaagactga-3′ (SEQ ID NO: 10); 5′-agtaacgcatggctgcggatgcta-3′(SEQ ID NO: 11). These lines were advanced to the T3 population and were found to be phenotypically uniform for kanamycin resistance and positive for PCR screening.

(iii) Southern and Northern Analysis: Southern- and northern-hybridizations were carried out with approximately 10 μg of genomic DNA and 30 μg of total RNA per lane. Genomic DNA was isolated from transgenic lines and wild-type Arabidopsis as described by Murray and Thompson (1980) and digested with HindIII, then transferred to a Hybond-N⁺ nylon membrane (Amersham, Buckinghamshire, UK). Southern hybridizations (65° C.) were carried out as described by Sambrook et al. (1989). DNA probes were labeled with [³²P]dCTP according to the manufacturer's instruction (Sigma, St. Louis). For Northern blot hybridization, total RNA was extracted from transgenic lines and wild-type Arabidopsis according to the method of Chomczynski and Sacci (1987). The probes and method of hybridization (55° C.) are the same with Southern blots.

### Example 1

### Comparison of the Amino Acid Sequences of Different DHN1s in Cultivated and Wild Barley

The Dhn1 gene coding region of wild barley was obtained from an accession (20-05) collected in Sede Boqer, located in the Negev desert in southern Israel, where the annual rain fall is normally less than 100 mm. The amino acid sequence predicted from Dhn1 in 20-05 was compared with other DHN1s of barley cultivars: Dicktoo (Genebank Accession No. 4105103) (SEQ ID NO: 3), Himalaya137 (Genebank Accession No 296198) (SEQ ID NO: 4), Hirnalaya139 (Genebank Accession No 118483) (SEQ ID NO: 5), and Georgie139 (Genebank Accession No CAA66970) (SEQ ID NO: 6).

The results depicted in FIG. 2A indicate that the DNH1 of the wild barley (20-05) presents two amino-acid substitutions in comparison to the barley cultivars: one in the S segment (S replaced by P) and the other one in the Φ segment (T replaced by M). Overall, as shown in the cladogram of FIG. 2B, DHN1 in wild barley (20-05) resembles barley cv Himalaya137 most closely, both having two S residues absent in the S segment (shown by the arrow) and a G substituted by D in the Y segment.

### Example 2

### Effect of Drought Stress on Transgenic Plants Overexpressing DHN1

Arabidopsis plants were transformed with the vector containing the dhn1 gene under the control of the CaMV 35S promoter (pNEdh construct) by the floral dip transformation protocol as described in Materials and Methods. Preliminary data were obtained with T2 plants (selected on hygromycin resistance) with drought and salt stress.

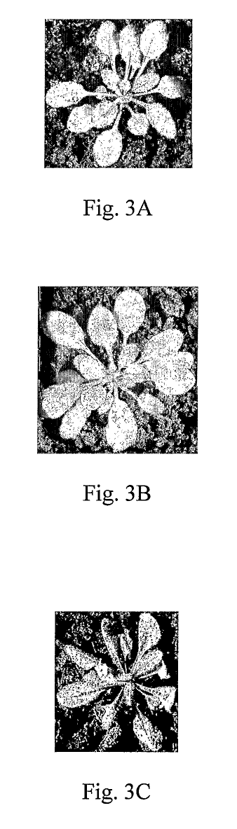

To test the drought stress, plants (control and transgenic) were grown in a greenhouse for 3 weeks with normal watering, followed by withholding water for 5 weeks. As shown in FIG. 3, under drought stress, the transgenic plants overexpressing DHN1 (FIG. 3B) are more drought tolerant than wild-type (control) plants (FIG. 3C). The transgenic plant under drought stress behaved like the wild-type plant not subjected to drought stress (FIG. 3A). DHN1 seems to have a protective role as anthocyanins were accumulated in the leaf tissue of the wild-type plants under water stress (FIG. 3C). Moreover, the flowering time in wild-type plants were 15 days earlier than that of the transgenic plants under the same water stress conditions (FIG. 3).

To test the effect of salt during the vegetative stage, two-week-old seedlings growing in the soil were irrigated with 0, 50, 75, 100, 150, 200, and 250 mM NaCl, respectively. Similar results were also obtained with salt tolerance, in respect of the early flowering and anthocyanin accumulation in wild-type plants compared with that of the transgenic plants (data not shown).

### Example 3

### Effect of Severe Drought Stress and Re-Watering on Transgenic Plants Overexpressing DHN1

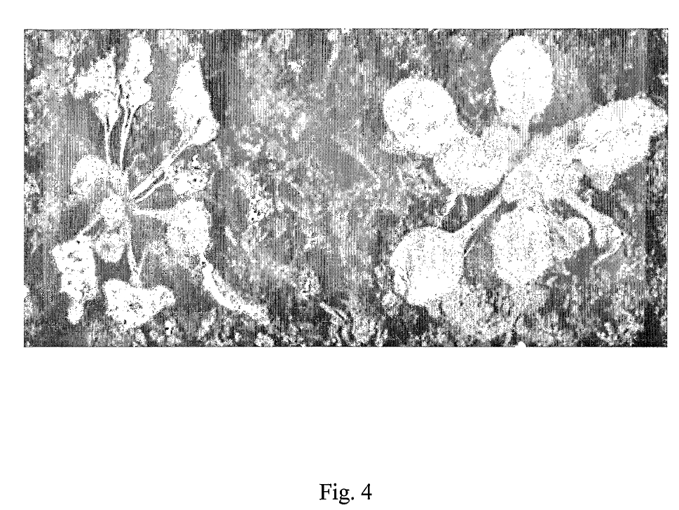

In order to test the plants under more severe drought stress, the transgenic (line N9) and wild-type plants were transferred to dry soil to undergo even stronger water stress. Eight-week old plants with normal watering were transplanted to dry soil and re-watered 24 hours after transplanting. The results are shown in FIG. 4. The photograph was taken 36 hours after re-watering. Both the transgenic (right) and the wild-type (left) plants wilted after transplanting. However, the N9 plant recovered and grew healthy following re-watering 24 hours after transplanting, whereas the wild-type plant (left) died consequently.

### REFERENCES

Asghar R, Fenton R D, DeMason D A, Close T J. Nuclear and cytoplasmic localization of maize embryo and aleurone dehydrin. Protoplasma 177:87-94(1994)

Badr A, Muller K, Schafer-Pregl R, El Rabey H, Effgen S, Ibrahim H H, Pozzi C, Rohde W, Salamini F. On the origin and domestication history of Barley (Hordeum vulgare). Mol Biol Evol. 17(4):499-510 (2000).

Baek H J, Beharav A, Nevo E. Ecological-genomic diversity of microsatellites in wild barley, Hordeum spontaneum, populations in Jordan. Theor Appl Genet. 106(3):397-410 (2003).

Barcelo P and Lazzeri P A. ‘Transformation of cereals by microprojectile bombardment of immature inflorescence and scutellum tissues’, in Methods in Molecular Biology, H. Jones, ed. 1995, Humana Press Inc.: Totowa, N.J., USA. pp. 113-22.

Becker D, Brettschneider R and Lorz H. Fertile transgenic wheat from microparticle bombardment of scutellar tissue. Plant Journal 5(2):299-307 (1994).

Becker D, Kemper E, Schell J, Masterson R. New plant binary vectors with selectable markers located proximal to the left T-DNA border. Plant Mol Biol 20:1195-1197(1992).

Borovskii G B, Stupnikova I V, Antipina A I, Vladimirova S V and Voinikov V K. Accumulation of dehydrin-like proteins in the mitochondria of cereals in response to cold, freezing, drought and ABA treatment. BMC Plant Biology. 2:5 (2002).

Campbell S A, Close T J. Dehydrins: genes, proteins, and associations with phenotypic traits. New Phytol. 137:61-74 (1997).

Cheng M, Fry J E, Pang S, Zhou H, Hironaka C M, Duncan D R, Conner T W and Wan Y, Genetic transformation of wheat mediated by Agrobacterium tumefaciens. Plant Physiology 115: 971-80 (1997).

Choi D W, Koag M C, Close T J. Map location of barley Dhn gene determined by gene-specific PCR. Theor Appl Genet. 101: 350-354 (2000).

Choi D W, Zhu B, Close T J. The barley (Hordeum vulgare L.) Dehydrin multigene family: sequences, allele types, chromosome assignments, and expression characteristics of 11 Dhn genes of cv Dicktoo. Theor Appl Genet 98: 1234-1247 (1999).

Chomczynski P and Sacci N. Single-step method of RNA isolation by acid guanidinium thiocyanate-phenol-chloroform extraction. Anal Biochem 162: 156-159 (1987).

Close T J. Dehydrins: a commonalty in the response of plants in dehydration and low temperature. Physiol Plant 100: 291-296 (1997).

Close T J. Dehydrins: emergence of a biochemical role of a family of plant dehydration proteins. Physiol Plant 97:795-803 (1996).

Close T J, Lammers P J. An osmotic stress protein of cyanobacteria is immunologically related to plant dehydrins. Plant Physiol. 101:773-779 (1993).

Close T J, Fenton R D, and F. Moonan. A view of plant dehydrins using antibodies specific to the carboxy terminal peptide. Plant Mol Biol 23:279-286 (1993).

Clough S J, Bent A F. Floral dip: a simplified method for Agrobacterium-mediated transformation of,Arabidopsis thaliana. Plant J 16: 735-743(1998).

Cushman J C, Bohnert H J. Genomic approaches to plant stress tolerance. Current Opinion in Plant Biology, 3:117-24 (2000).

Danyluk J, Perron A, Houde M, Limin A, Fowler B, Benhamou N, and F. Sarhan. Accumulation of an acidic dehydrin in the vicinity of the plasma membrane during cold acclimation of wheat. Plant Cell. 10:623-638 (1998).

Egerton-Warburton L M, Balsamo R A, Close T J. Temporal accumulation and ultrastructural localization of dehydrins in Zea mays L. Physiol Plant 101: 545-555 (1997).

Gelvin S B and Schilperoort R A (Eds), Plant Molecular Biology Manual, 2nd Edition, Kluwer Academic Press, Dordrecht, Netherlands (1994).

Harwood W A, Ross S M, Cilento P, Snape J W. The effect of DNA/gold particle preparation technique, and particle bombardment device, on the transformation of barley (Hordeum vulgare). Euphytica 111: 67-76 (2000).

Jaglo-Ottosen K R, Gilmour S J, Zarka D G, Schabenberger O, Thomashow M F. Arabidopsis CBF1 overexpression induces COR genes and enhances freezing tolerance. Science 280:104-6 (1998).

Kasuga M, Liu Q, Miura S, Yamaguchi-Shinozaki K and Shinozaki K. Improving plant drought, salt, and freezing tolerance by gene transfer of a single stress-inducible transcription factor. Nature Biotech 17: 287-291 (1999).

Kaye C, Neven L, Hofig A, Li Q B, Haskell D, and C. Guy. Characterization of a gene for spinach CAP160 and expression of two spinach cold-acclimation proteins in tobacco. Plant Physiol 116(4): 1367-1377 (1998).

Koag M C, Fenton R D, Wilkens S, and T J Close. The binding of maize DHN1 to lipid vesicles. Gain of structure and lipid specificity. Plant Physiol. 131: 309-316 (2003).

Li R, Brawley S H, Close T J: Proteins immunologically related to dehydrins in fucoud algae. J Phycol. 34:642-650 (1998).

Liu Q, Kasuga M, Sakuma Y, Abe H, Miura S, et al. Two transcription factors, DREB1 and DREB2, with an EREBP/AP2 DNA-binding domain separate two cellular signal transduction pathways in drought- and low temperature-responsive gene expression, respectively, in Arabidopsis. Plant Cell 10:1391-406 (1998).

Mtwisha L, Brandt W, McCready S, Lindsey G G. HSP 12 is a LEA-like protein in Saccharomyces cerevisiae. Plant Mol Biol. 37:513-521(1998).

Murray M G, Thompson W F. Rapid isolation of high molecular weight plant DNA Nucleic Acids Res. 8:4321-5 (1980).

Nehra N S, Chibbar R N, Leung N, Caswell K, Mallard C, Steinhauer L, Baga M and Kartha K. Self-fertile transgenic wheat plants regenerated from isolated scutellar tissues following microprojectile bombardment with two distinct gene constructs. Plant Journal 5(2): 285-97 (1994).

Nevo, E. Origin, evolution, population genetics and resources for breeding of wild barley, Hordeum spontaneum, in the Fertile Crescent. In: Barley: Genetics, Mol Biol Biotech, Shewry, P. (ed.). C.A.B. International, pp. 19-43. (1992).

Pan A, Hayes P M, Chen F, Chen T H H, Blake T, Wright S, Karsai I, and Z. Bedo. Genetic analysis of the components of winter hardiness in barley (Hordeum vulgare L.) Theor Appl Genet 89: 900-910 (1994).

Sambrook J, Fritsch F F and Maniatis T. Molecular Cloning: A Laboratory Manual, 2d edition, Cold Spring Harbor Laboratory Press, Cold Spring Harbor, N.Y. (1989).

Song W and Henry R J. Molecular analysis of the DNA polymorphism of wild barley (Hordeum spontaneum) germplasm by the polymerase chain reaction. Genetic Resources and Crop Evolution. 42: 273-281(1995).

Tingay S, McElroy D, Kalla R, Fieg S, Wang M, Thornton S and Brettel R, Agrobacterium tumefaciens-mediated barley transformation. Plant Journal 11: 1369-76 (1997).

Turpeinen T, Tenhola T, Manninen O, Nevo E, Nissila E. Microsatellite diversity associated with ecological factors in Hordeum spontaneum populations in Israel. Mol Ecol. 0(6):1577-91 (2001).

Wan Y and Lemaux P G. Generation of large numbers of independently transformed fertile barley plants. Plant Physiology 104: 37-48 (1994).

Weeks J T, Andersen O D and Blechl A E, Rapid production of multiple independent lines of fertile transgenic wheat (Triticuin aestivum L.). Plant Physiology 102(4): 1077-84 (1993).

Welin B V, Olson A, Nylander M and E T Palva. Characterization and Differential Expression of Dhn/Lea/Rab-Like Genes During Cold-Acclimation and Drought Stress in Arabidopsis thaliana. Plant Mol. Biol. 26(1): 131-144 (1994).

Weining S and Langridge P. Identification and mapping of polymorphisms in cereals based on the polymerase chain reaction. Theor Appl Genet 82: 209-216 (1991).

Wise M J and Tunnacliffe A. POPP the question: what do LEA proteins do? Trends in Plant Science, 9, 13-17 (2004).

Zhang L, Ohta A, Takagi M, Imai R. Expression of plant group 2 and group 3 LEA genes in Saccharomyces cerevisiae revealed functional divergence among LEA proteins. J Biochem. 127: 611-616 (2000).

Zhu B, Choi D W, Fenton R, and Close T J. Expression of the barley dehydrin multigene family and the development of freezing tolerance. Mol. Gen. Genet. 264:145-153 (2000).

1. An isolated DNA having a nucleotide sequence coding for the dehydrin 1 (DHN1) protein of SEQ ID NO:2.

2. An isolated DNA according to claim 1, having a nucleotide sequence selected from the group consisting of SEQ ID NO: 1 and a DNA having a nucleotide sequence differing from SEQ. ID NO: 1 in codon sequence due to the degeneracy of the genetic code.

3. An isolated DNA according to claim 2 comprising a DNA consisting of the nucleotide sequence of SEQ ID NO: 1, said DNA comprising the coding region of the dehydrinl gene (dhn1) coding for the dehydrin 1 (DHN1) protein of SEQ ID NO: 2.

4. An isolated DNA according to claim 1 comprising a cDNA consisting of the nucleotide sequence coding for the dehydrin 1 (DHN1) protein of SEQ ID NO: 2.

5. A chimeric DNA construct capable of expression in plant cells, comprising: (a) a DNA sequence of SEQ ID NO: 1 coding for the DHN1 protein of SEQ ID NO: 2 or a cDNA coding for the DHN1 protein of SEQ ID NO: 2, and (b) DNA sequences enabling expression of the DHN1 protein in plant cells.

6. A chimeric DNA construct according to claim 5, comprising (a) a DNA sequence of SEQ ID NO: 1 and (b) DNA sequences enabling expression of the DHN1 protein in plant cells.

7. A recombinant vector comprising the DNA according claim 1.

8. An expression vector comprising the chimeric DNA construct of claim 5.

9. A transgenic plant transformed with the DNA of SEQ ID NO: 1 or cDNA coding for the protein DHN1 of SEQ ID NO: 2, said DNA being operably linked to DNA sequences enabling expression of the DHN1 protein in plant cells and subsequent improvement of tolerance of the plant to abiotic stresses.

10. A transgenic plant which contains in its cells a chimeric DNA according to claim 6 such that the plant exhibits tolerance to abiotic stresses.

11. A transgenic plant according to claim 9 which is a cereal plant.

12. A transgenic plant according to claim 11 wherein said cereal is barley.

13. A transgenic plant according to claim 9 wherein said abiotic stress is drought, cold/freezing or high salinity stress.

14. A recombinant vector comprising the chimeric DNA construct of claim 5.

15. A recombinant vector comprising the chimeric DNA construct of claim 6.

16. An expression vector comprising the chimeric DNA construct of claim 6.

17. A transgenic plant according to claim 10 which is a cereal plant.

18. A transgenic plant according to claim 17 wherein said cereal is barley.

19. A transgenic plant according claim 10 wherein said abiotic stress is drought, cold/freezing or high salinity stress.
